(19) United States
(12) Patent Application Publication
(10) Pub. No.: US 2026/0137878 A1
(43) Pub. Date: May 21, 2026

(54) SYSTEM AND METHOD FOR OPTIMIZING INSUFFLATION PRESSURE DURING LAPAROSCOPIC SURGICAL PROCEDURES TO IMPROVE PATIENT OUTCOMES

(71) Applicant: Conmed Corporation, Largo, FL (US)

(72) Inventors: Joseph Allen, Broomfield, CO (US); Mahesh Krishnamoorthy, Parker, CO (US); Sami Al-Abed, Glendale, CO (US)

(73) Assignee: Conmed Corporation, Largo, FL (US)

(21) Appl. No.: 19/120,000

(22) PCT Filed: Nov. 3, 2023
(86) PCT No.: PCT/US2023/036746

§ 371 Date: Apr. 10, 2025

Related U.S. Application Data
(60) Provisional application No. 63/423,570, filed on Nov. 8, 2022.

(51) Int. Cl. A61M 13/00

(52) U.S. Cl. CPC A61M 13/003; A61M 2205/3334; A61M 2205/3344; A61M 2205/3592; A61M 2205/52; A61M 2230/30; A61M 2230/432

(57) ABSTRACT

A method of optimizing insufflation pressure during a laparoscopic surgical procedure that includes logging insufflation data during a type of laparoscopic surgical procedure performed on a plurality of patients over a period of time, correlating the insufflation data with one or more insufflation parameters having an impact on surgical outcomes for the type of laparoscopic surgical procedure, determining an optimal value of insufflation pressure for the type of laparoscopic surgical procedure based upon one or more of the insufflation parameters, and providing guidance for operating an insufflation device during the type of laparoscopic surgical procedure performed on a patient at the optimal value of insufflation pressure to improve the surgical outcome for that patient.

### CROSS-REFERENCE TO RELATED APPLICATION

This application claims the benefit of priority to U.S. Provisional patent application Ser. No. 63/423,570, filed Nov. 8, 2022, the disclosure of which is incorporated herein by reference in its entirety.

### BACKGROUND OF THE DISCLOSURE

### 1. Field of the Disclosure

The subject disclosure is directed to laparoscopic surgery, and more particularly, to a system and method for optimizing insufflation pressures during a laparoscopic surgical procedure by logging and analyzing insufflation data over a period of time to improve patient outcomes.

### 2. Description of Related Art

Laparoscopic or “minimally invasive” surgical techniques have become commonplace in the performance of procedures such as cholecystectomies, appendectomies, hernia repair and nephrectomies. Such procedures commonly involve filling or “insufflating” the abdominal cavity with a pressurized fluid, such as carbon dioxide, to create an operating space, which is referred to as a pneumoperitoneum.

Insufflators are used to deliver pressurized insufflation gas to a patient's abdominal cavity during a surgical procedure, including those designed to recirculate insufflation gas through a recirculation circuit to maintain a stable pneumoperitoneum, as disclosed for example in commonly assigned U.S. Pat. Nos. 9,067,030 and 9,375,539, as well as U.S. Patent Application Publication 2022/0233891, the disclosures of which are incorporated herein by reference.

Current insufflators known in the art, including those designed to recirculate insufflation gas, are not presently adapted and configured to collect and/or record insufflation data relating to the performance of laparoscopic surgical procedures. Such insufflation data could be collected from a plurality of patients over a period of time for certain surgical procedures and then analyzed or otherwise correlated with certain known insufflation operating parameters to enable the optimization of insufflation pressures and thereby improve patient outcomes. Moreover, it is known that patient outcomes will improve by lowering the insufflation set pressure for a given surgical procedure. For example, lower insufflation pressure will reduce the risk of factors related to the occurrence of subcutaneous emphysema during laparoscopic surgery.

The subject application provides a system and method for optimizing insufflation pressures during a laparoscopic surgical procedure by logging and analyzing insufflation data over a period of time to improve patient outcomes. Moreover, the data analysis enables a clinician to optimize permutations of insufflation parameters and avoid insufflation parameter set points that have been proven to produce adverse patient outcomes.

### SUMMARY OF THE DISCLOSURE

The subject disclosure is directed to a new and useful method of optimizing insufflation pressure during a laparoscopic surgical procedure. The method includes the steps of: logging insufflation data during a type of laparoscopic surgical procedure performed on a plurality of patients over a period of time; correlating the insufflation data with one or more insufflation parameters having an impact on surgical outcomes for the type of laparoscopic surgical procedure; determining an optimal value of insufflation pressure for the type of laparoscopic surgical procedure based upon one or more of the insufflation parameters; and providing guidance for operating an insufflation device during the type of laparoscopic surgical procedure performed on a patient at the optimal value of insufflation pressure to improve the surgical outcome for that patient.

The method further includes the step of associating the insufflation data with a particular surgeon or surgical facility, and time stamping and/or encrypting the insufflation data to protect patient confidentiality. In one embodiment, the insufflation data is logged on an insufflation device having an embedded data logging module within its circuitry, and the method includes the step of transferring the insufflation data from the insufflation device to an external storage device by way of a wired or a wireless communication link.

Preferably, one or more algorithms are used for correlating the insufflation data with one or more of the insufflation parameters, and one or more algorithms are used for determining the optimal value of insufflation pressure. Preferably, the insufflation data includes at least one of a set pressure, an operating pressure, a total volume of insufflation gas used during the surgical procedure, gas concentration measurements, and gas flow measurements. Preferably, the insufflation parameters include at least one intra-operative pain indices, post-operative pain indices, inter-operative hemodynamic pressure, end-tidal CO₂, mean blood pressure, and peritoneal absorption of CO₂. It is further envisioned that the optimal pressure value is at least one of a pre-operative pressure value, an intra-operative pressure value, and a post-operative pressure value.

The method further includes the step of combining the insufflation data with data logged by at least one other device or system used during a laparoscopic surgical procedure. For example, the insufflation data can be combined with data logged by at least one of an anesthesia hemodynamic monitoring system, a surgical visualization system, and a robotic surgical system.

The subject disclosure is also directed to a system for optimizing insufflation pressure during a laparoscopic surgical procedure over a period of time, which includes an insufflation device having a module for storing insufflation data logged from one or more sensors during a type of laparoscopic surgical procedure performed on a plurality of patients over a period of time, and a communication link operatively associated with the insufflation device for transmitting the insufflation data to a processor.

The processor is adapted and configured to correlate the insufflation data with one or more insufflation parameters having an impact on surgical outcomes for the type of laparoscopic surgical procedure, to determine an optimal value of insufflation pressure at which to operate the insufflation device for the type of laparoscopic surgical procedure to improve the surgical outcome for a patient.

The insufflation data includes at least one of a set pressure, an operating pressure, a total volume of insufflation gas used during the surgical procedure, gas concentration measurements, and gas flow measurements. The insufflation parameters include at least one of intra-operative pain indices, post-operative pain indices, inter-operative hemodynamic pressure, end-tidal CO₂, mean blood pressure, and peritoneal absorption of CO₂. The optimal pressure value is at least one of a pre-operative pressure value, an intra-operative pressure value, and a post-operative pressure value.

These and other features of the system and method of the subject disclosure will become more readily apparent from the following detailed description of the disclosed embodiments taken in conjunction with the drawings.

### BRIEF DESCRIPTION OF THE DRAWINGS

So that those skilled in the art will readily understand how to make and use the system and method of the subject disclosure without undue experimentation, preferred embodiments thereof will be described in detail herein below with reference to the figures wherein:

FIG. 1 is a schematic representation of an insufflation device adapted and configured for logging and transferring insufflation data in accordance with the subject disclosure; and

FIG. 2 is a flow chart illustrating the operational steps of the method of optimizing insufflation pressures during a laparoscopic surgical procedure in accordance with the subject disclosure.

### DETAILED DESCRIPTION OF THE PREFERRED EMBODIMENTS

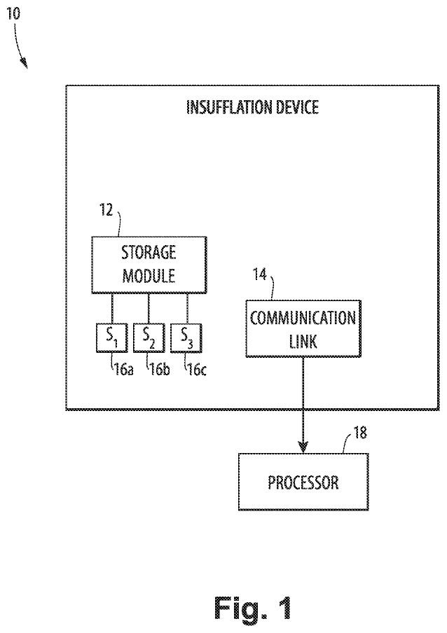

Referring now to the drawings, there is illustrated in FIG. 1 a new and useful surgical gas delivery system designated generally by reference numeral 10 which is adapted and configured to optimize insufflation pressure during a laparoscopic surgical procedure. The insufflation device 10 can be a multi-modal insufflation and recirculation device, such the AirSeal® iFS access management platform manufactured and sold by Conmed Corporation of Largo, FL.

The insufflation device 10 has a storage module 12 embedded within its circuitry for storing insufflation data logged from one or more sensors 16a-16c during a type of laparoscopic surgical procedure performed on a plurality of patients over a period of time (e.g., a month or a year). For example, insufflation data can be logged during the performance of procedures such as cholecystectomies, appendectomies, hernia repair and nephrectomies, which may or may not be robotically assisted.

Sensors 16a-16c can include at least one of a pressure sensor, a flow sensor, a temperature sensor and/or a gas quality sensor. The insufflation device further includes a communication link 14 for transmitting the insufflation data to a processor 18. The communication link 14 could be a wireless communication link or a hard-wired communication link, such as a data transmission cable. Alternatively, the insufflation device 10 could have a data port for receiving a USB drive or another form of portable data storage device for downloading the logged insufflation data.

The processor 18 is adapted and configured to correlate the insufflation data with one or more insufflation parameters having an impact on surgical outcomes for the type of laparoscopic surgical procedure, to determine an optimal value of insufflation pressure at which to operate the insufflation device for the type of laparoscopic surgical procedure to improve the surgical outcome for a patient.

The types of insufflation data preferably include, but are not limited to, at least one of a set pressure, an operating pressure, a total volume of insufflation gas used during the surgical procedure, gas concentration measurements, and gas flow measurements. The types of insufflation parameters preferably include, but are not limited to, at least one of intra-operative pain indices, post-operative pain indices, inter-operative hemodynamic pressure, end-tidal CO₂, mean blood pressure, and peritoneal absorption of CO₂. The type of optimal pressure value is at least one of a pre-operative pressure value, an intra-operative pressure value, and a post-operative pressure value, and the like.

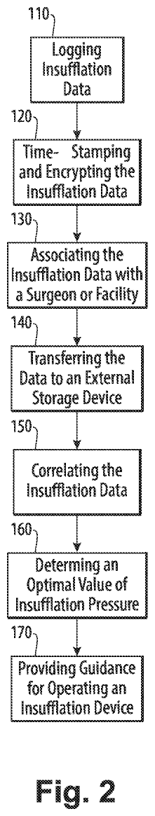

Referring to FIG. 2, the subject disclosure is directed to a new and useful method of optimizing insufflation pressure during a laparoscopic surgical procedure, which is designated generally by reference numeral 100. The method includes step 110 when insufflation data is logged by the insufflation device 10 during a type of laparoscopic surgical procedure performed on a plurality of patients over a period of time. This could be done over the course of a day(s), month(s), or year(s), for a particular surgeon or for a particular surgical facility, to map out trends over time.

At step 120, the method involves time stamping and/or encrypting the insufflation data. This is done to organize and protect the confidentiality of patient data. Then, at step 130, the insufflation data may be associated with a particular surgeon or a particular surgical facility, such as a hospital or a clinic. At step 140, the insufflation data is transferred from the insufflation device 10 to an external storage device. The external storage device could be co-located with the insufflation device 10 or remotely located. This data transfer may be done by way of a wired communication link or a wireless communication link.

Then, at step 150 the insufflation data is correlated with one or more insufflation parameters having an impact on surgical outcomes for the type of laparoscopic surgical procedure. Thereafter, at step 160, the method involves determining an optimal value of insufflation pressure for the type of laparoscopic surgical procedure based upon one or more of the insufflation parameters. At step 170, the method involves providing guidance for operating an insufflation device during the type of laparoscopic surgical procedure performed on a patient at the optimal value of insufflation pressure to improve the surgical outcome for that patient.

For example, guidance on the optimum insufflation set pressure for a certain procedure (e.g., a laparoscopic hernia repair) to be performed on a particular patient (e.g., a 65 year old male) having a particular set of physical characteristics (e.g., Body Mass Index>30) could be provided or otherwise delivered from the data processing entity to a surgeon or hospital facility utilizing the insufflation device 10, prior to the procedure. Thereupon, the operating room staff could set the insufflation pressure on the insufflation device according to that guidance to ensure a desirable surgical outcome.

Alternatively, the data processing entity could provide or otherwise distribute guidance in the form of a table of optimum pressure settings for certain surgical procedures to be performed on particular types of patients having particular sets of physical characteristics. This information could be provided to interested clinicians as a service to notify them of trends, enabling them to fine-tune their usage of the insufflation device. The service could be free or fee-based, creating a profit center for the device manufacturer.

It is envisioned that one or more algorithms are used for correlating the insufflation data with one or more of the insufflation parameters, and one or more algorithms are used for determining the optimal value of insufflation pressure. The algorithms employed in this method can rely upon predictive analytics or machine learning techniques, and involve linear regression models, clustering models, classification models, time-series models and/or combinations thereof.

Preferably, the insufflation data includes at least one of a set pressure, an operating pressure, a total volume of insufflation gas used during the surgical procedure, gas concentration measurements, and gas flow measurements. Preferably, the insufflation parameters include at least one of intra-operative pain indices, post-operative pain indices, inter-operative hemodynamic pressure, end-tidal CO₂, mean blood pressure, and peritoneal absorption of CO₂. It is further envisioned that the optimal pressure value is at least one of a pre-operative pressure value, an intra-operative pressure value, and a post-operative pressure value.

The method further includes the step of combining the insufflation data with data logged by at least one other device or system used during a laparoscopic surgical procedure. For example, the insufflation data can be combined with data logged by at least one of an anesthesia hemodynamic monitoring system, a surgical visualization system, and a robotic surgical system.

While the subject disclosure has been shown and described with reference to preferred embodiments, those skilled in the art will readily appreciate that changes and/or modifications may be made thereto without departing from the spirit or scope of the subject disclosure.

What is claimed is:

1. A method of optimizing insufflation pressure during a laparoscopic surgical procedure comprising the steps of:
a) logging insufflation data during a type of laparoscopic surgical procedure performed on a plurality of patients over a period of time;
b) correlating the insufflation data with one or more insufflation parameters having an impact on surgical outcomes for the type of laparoscopic surgical procedure;
c) determining an optimal value of insufflation pressure for the type of laparoscopic surgical procedure based upon one or more of the insufflation parameters; and
d) providing guidance for operating an insufflation device during the type of laparoscopic surgical procedure performed on a patient at the optimal value of insufflation pressure to improve the surgical outcome for that patient.

2. The method according to claim 1, further comprising the step of associating the insufflation data with a particular surgeon or surgical facility.

3. The method according to claim 1, further comprising the steps of time stamping and encrypting the insufflation data.

4. The method according to claim 1, wherein the insufflation data is logged on an insufflation device having an embedded data logging module within its circuitry.

5. The method according to claim 1, further comprising the step of transferring the insufflation data from the insufflation device to an external storage device by way of a wired or a wireless communication link.

6. The method according to claim 1, wherein one or more algorithms are used for correlating the insufflation data with one or more of the insufflation parameters.

7. The method according to claim 1, wherein one or more algorithms are used for determining the optimal value of insufflation pressure.

8. The method according to claim 1, wherein the insufflation data includes at least one of a set pressure, an operating pressure, a total volume of insufflation gas used during the surgical procedure, gas concentration measurements, and gas flow measurements.

9. The method according to claim 1, wherein the insufflation parameters include at least one of intra-operative pain indices, post-operative pain indices, inter-operative hemodynamic pressure, end-tidal CO₂, mean blood pressure, and peritoneal absorption of CO_{2.}

10. The method according to claim 1, wherein the optimal pressure value is at least one of a pre-operative pressure value, an intra-operative pressure value, and a post-operative pressure value.

11. The method according to claim 1, further comprising the step of combining the insufflation data with data logged by at least one other device or system used during a laparoscopic surgical procedure.

12. The method according to claim 11, wherein the insufflation data is combined with data logged by at least one of an anesthesia hemodynamic monitoring system, a surgical visualization system, and a robotic surgical system.

13. A system for optimizing insufflation pressure during a laparoscopic surgical procedure comprising:
a) an insufflation device having a module for storing insufflation data logged from one or more sensors during a type of laparoscopic surgical procedure performed on a plurality of patients over a period of time; and
b) a communication link operatively associated with the insufflation device for transmitting the insufflation data to a processor that is configured to correlate the insufflation data with one or more insufflation parameters having an impact on surgical outcomes for the type of laparoscopic surgical procedure, to determine an optimal value of insufflation pressure at which to operate the insufflation device for the type of laparoscopic surgical procedure to improve the surgical outcome for a patient.

14. The system as recited in claim 13, wherein the insufflation data includes at least one of a set pressure, an operating pressure, a total volume of insufflation gas used during the surgical procedure, gas concentration measurements, and gas flow measurements.

15. The system as recited in claim 13, wherein the insufflation parameters include at least one of intra-operative pain indices, post-operative pain indices, inter-operative hemodynamic pressure, end-tidal CO₂, mean blood pressure, and peritoneal absorption of CO₂.

16. The system as recited in claim 13, wherein the optimal pressure value is at least one of a pre-operative pressure value, an intra-operative pressure value, and a post-operative pressure value.
